(19) United States
(12) Patent Application Publication
(10) Pub. No.: US 2024/0397945 A1
(43) Pub. Date: Dec. 5, 2024

(54) PRESERVATION SOLUTION OF MAGNETIC BEADS AND PRESERVATION METHOD OF MAGNETIC BEADS

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Zijian ZHAO, Beijing (CN); Ding DING, Beijing (CN); Siyi YIN, Beijing (CN)

(73) Assignees: BEIJING BOE TECHNOLOGY DEVELOPMENT CO., LTD., Beijing (CN); BOE TECHNOLOGY GROUP CO., LTD., Beijing (CN)

(21) Appl. No.: 18/262,657

(22) PCT Filed: Jul. 4, 2022
(86) PCT No.: PCT/CN2022/103699

§ 371 Date: Jul. 24, 2023

(30) Foreign Application Priority Data
Jul. 28, 2021 (CN) 202110857803.9

(51) Int. Cl. A01N 37/46; A01N 25/02; A01N 25/30; A01N 37/18; A01N 37/20; A01P 1/00; A61L 101/40

(52) U.S. Cl. CPC A01N 37/46; A01N 25/02; A01N 25/30; A01N 37/18; A01N 37/20; A01P 1/00; A61L 2101/40

(57) ABSTRACT

A preservation solution of magnetic beads includes a surfactant, a water-soluble salt and a bacteriostatic agent.

### CROSS-REFERENCE TO RELATED APPLICATIONS

This application is a national phase entry under 35 USC 371 of International Patent Application No. PCT/CN2022/103699, filed on Jul. 4, 2022, which claims priority to Chinese Patent Application No. 202110857803.9, filed on Jul. 28, 2021, which are incorporated herein by reference in their entireties.

### TECHNICAL FIELD

The present disclosure relates to the field of biomedicine technologies, in particular, to a preservation solution of magnetic beads and a preservation method of magnetic beads.

### BACKGROUND

Magnetic beads play an extremely important role in the whole life science field. As a key raw material, magnetic beads play a vital role in various sub-fields such as immunity, pathology, physiology, pharmacology, microbiology, biochemistry and molecular genetics, and are increasingly widely used in immune detection, cell separation, biological macromolecule purification and molecular biology.

### SUMMARY

In an aspect, a preservation solution of magnetic beads is provided. The preservation solution of magnetic beads includes a surfactant, a water-soluble salt and a bacteriostatic agent.

In some embodiments, the water-soluble salt is selected from one of divalent salts or a plurality of combinations of the divalent salts, and in a case where the water-soluble salt is selected from the plurality of combinations of the divalent salts, a chemical reaction does not occur between a plurality of divalent salts.

In some embodiments, the water-soluble salt is selected from one of calcium salts and magnesium salts or a plurality of combinations of the calcium salts and the magnesium salts.

In some embodiments, the water-soluble salt is selected from one or a combination of calcium chloride and magnesium chloride.

In some embodiments, a molar concentration of the water-soluble salt in the preservation solution is in a range of 1 mol/L to 3 mol/L, inclusive.

In some embodiments, the bacteriostatic agent is selected from antibiotics.

In some embodiments, the antibiotics are broad-spectrum antibiotics.

In some embodiments, an antibiotic is selected from one of or a plurality of combinations of chloramphenicol, tetracycline and vancomycin, and a volume percentage of each antibiotic in the preservation solution is in a range of 0.1% to 0.5%, inclusive.

In some embodiments, a volume percentage of the surfactant in the preservation solution is in a range of 0.5% to 5%, inclusive.

In some embodiments, the surfactant includes an ionic surfactant or a nonionic surfactant.

In some embodiments, the surfactant is selected from any or combinations of two or more of polysorbate-20, sodium dodecyl sulfate and sorbitan fatty acid esters.

In some embodiments, the preservation solution of magnetic beads further includes ethylenediaminetetraacetic acid.

In some embodiments, a molar concentration of the ethylenediaminetetraacetic acid in the preservation solution is in a range of 0.5 mmol/L to 20 mmol/L, inclusive.

In some embodiments, the preservation solution of magnetic beads further includes a buffer agent, an additive amount of the buffer agent causes a pH value of the preservation solution is in a range of 7.5 to 8.5, inclusive.

In some embodiments, a molar concentration of the buffer agent in the preservation solution is in a range of 0.1 mmol/L to 10 mmol/L, inclusive.

In another aspect, a preservation method of magnetic beads is provided. The method includes:
dispersing the magnetic beads in the preservation solution of magnetic beads as described above.

In some embodiments, a preservation temperature of the magnetic beads is in a range of 4° C. to 8° C., inclusive.

### BRIEF DESCRIPTION OF THE DRAWINGS

In order to describe technical solutions in the present disclosure more clearly, accompanying drawings to be used in some embodiments of the present disclosure will be introduced briefly. Obviously, the accompanying drawings to be described below are merely accompanying drawings of some embodiments of the present disclosure, and a person of ordinary skill in the art can obtain other drawings according to those drawings.

FIG. 1A is a dispersion diagram of magnetic beads in a comparative example under a microscope after preservation for 6 months;

FIG. 1B is a dispersion diagram of magnetic beads in an experimental example 1 under a microscope after preservation for 6 months;

FIG. 2 is a broken line graph of variations of agglomeration ratio of magnetic beads in comparative example and experimental example 1 during preservation; and

FIG. 3 is a histogram of agglomeration ratio of magnetic beads after preservation for 3 months in experimental example 1 to experimental example 6.

### DETAILED DESCRIPTION

Technical solutions in some embodiments of the present disclosure will be described clearly and completely with reference to the accompanying drawings below. Obviously, the described embodiments are merely some but not all embodiments of the present disclosure. All other embodiments obtained by a person of ordinary skill in the art based on the embodiments of the present disclosure shall be included in the protection scope of the present disclosure.

Unless the context requires otherwise, throughout the description and the claims, the term “comprise” and other forms thereof such as the third-person singular form “comprises” and the present participle form “comprising” are construed as open and inclusive, i.e., “including, but not limited to”. In the description of the specification, the terms such as “one embodiment”, “some embodiments”, “exemplary embodiments”, “example”, “specific example” or “some examples” are intended to indicate that specific features, structures, materials or characteristics related to the embodiment(s) or example(s) are included in at least one embodiment or example of the present disclosure. Schematic representations of the above terms do not necessarily refer to the same embodiment(s) or example(s). In addition, the specific features, structures, materials, or characteristics described here may be included in any one or more embodiments or examples in any suitable manner.

The phrase “at least one of A, B and C” has a same meaning as the phrase “at least one of A, B or C”, and they both include the following combinations of A, B and C: only A, only B, only C, a combination of A and B, a combination of A and C, a combination of B and C, and a combination of A, B and C.

The phrase “A and/or B” includes the following three combinations: only A, only B, and a combination of A and B.

Biological magnetic beads (also referred to as nano magnetic beads) refer to super paramagnetic microspheres with a small particle size (generally in a range of 1 nm to 100 nm), and have super paramagnetic property, that is, the magnetic beads have strong magnetic responsiveness in an external magnetic field, and after the magnetic field is removed, the magnetism of the magnetic beads disappears immediately (i.e., there is no residual magnetism) and the magnetic beads are re-dispersed in the solution evenly. Utilizing this property, certain components in the liquid may be adsorbed, and then magnetic beads are separated through magnetic separation, so as to achieve the purpose of separating components.

In order to be able to adsorb the desired substances, outer surfaces of the magnetic beads must be wrapped with groups with specificity, such as amino groups, hydroxyl groups, carboxyl groups, sulfhydryl groups and other functional groups. The magnetic beads specifically bind to the target molecules through these groups, and then the magnetic beads are collected by magnetic force, so that the required substances may be separated.

Compared with traditional separation methods, the use of magnetic beads for the separation of complex components in biochemical samples may simultaneously achieve separation and enrichment, thereby effectively improving separation speed and enrichment efficiency, and greatly improving sensitivity of analysis and detection.

At present, magnetic beads have an application market of tens of billions of dollars, and many giant companies including Beckman, Spherotech, Invitrogen, Millipore-Sigma, etc. all regard the magnetic bead business as one of their main businesses. However, although the magnetic beads are widely used, preservation and transportation of the magnetic beads have always been a big problem. Magnetic beads are not resistant to low and high temperatures, and can only be transported at a temperature in a range of 4° C. to 8° C. Therefore, a preservation solution of magnetic beads requires a long shelf life and tolerance at this room temperature condition. In addition, magnetic beads are easy to aggregate, but the aggregated magnetic beads will have a huge and uncontrollable impact on separation and detection results. Therefore, long term and stable maintenance of monodispersion of magnetic beads is extremely important for the entire industry. Not only it may greatly reduce the cost of logistics and storage, but also provide extremely favorable conditions for downstream application products.

At present, the preservation solutions of magnetic beads sold on the market are generally aqueous solutions with surfactants and buffer agents added, and have a shelf life of only about ten days, and the preservation effect is poor. In addition, it has been found during use that with different specific groups on the surface of the magnetic beads, there are certain differences in preservation effect of the preservation solution, and the versatility is poor.

In light of this, some embodiments of the present disclosure provide a preservation solution of magnetic beads, which includes a surfactant, a water-soluble salt, a bacteriostatic agent, water, and the like.

Examples of the surfactant may include a cationic surfactant, an anionic surfactant, a zwitterionic surfactant, a nonionic surfactant, and the like. A part of the cationic surfactant playing a surface active role is cations, such as quaternary ammonium salts. A part of the anionic surfactant playing a surface active role is anions, such as sulfates. In a molecular structure of the zwitterionic surfactant, hydrophilic groups connected to a hydrophobic group are two groups with opposite electrical properties, that is, groups with positive and negative charges. For example, an anion part may be carboxylates, and a cationic part may be amine salts or quaternary ammonium salts. The nonionic surfactant do not dissociate in water, and in a molecular structure thereof, hydrophilic groups are mainly polyoxyethylene groups and hydroxyl groups of polyols, and lipophilic groups are mainly long-chain fatty acids or long-chain fatty alcohols, and alkyl groups or aromatic groups.

When the magnetic beads are mixed with the preservation solution, if the preservation solution does not contain a surfactant, the magnetic beads will be dispersed into a lot of tiny particles in the preservation solution, expanding the contact areas between the magnetic beads, leading to an increase in an energy potential of a system and resulting in an unstable state. In a case where the surfactant is added, lipophilic groups of the surfactant are adsorbed on a surface of hydrophobic chains of organic molecules on the magnetic bead, while hydrophilic groups thereof extend into the water and are arranged directionally on a surface of the magnetic bead to form a layer of hydrophilic molecular film, so that an interfacial tension between the magnetic bead and water is reduced, thereby lowering the energy potential of the system, reducing attractive force between the magnetic beads, and preventing the aggregation of the magnetic beads. The molecular film formed by the hydrophilic groups of the surfactant being arranged directionally on the surface of the magnetic bead is a solid protective film, which may prevent the magnetic beads from aggregating due to collision. If the surfactant is an ionic surfactant, the magnetic beads will carry the same charges, so that the mutual repulsion increases, thereby preventing the magnetic beads from aggregating during frequent collisions. Therefore, the addition of surfactant may keep a suspension of the magnetic beads stable, and keep the magnetic beads dispersed to a certain extent, thereby improving the adsorption efficiency of magnetic beads to biochemical samples (such as nucleic acid).

However, for different types of surfactants, the addition amounts in the preservation solution are also different. For the nonionic surfactant, the surfactant mainly disperses the magnetic beads through steric hindrance effect. For the ionic surfactant, the surfactant may disperse the magnetic beads through charge repulsion. However, the charge neutralization effect between the surfactant and the surfaces of the magnetic beads may destroy the electrostatic repulsion between the magnetic beads, so as to make dispersion of the magnetic beads destabilize and result in aggregation of the magnetic beads. Moreover, the magnetic beads will form coarse floccules due to a bridging effect of the polymer surfactant (i.e., the flocculation of surfactant), especially in a case where of a large addition amounts of the surfactant. Thus, it is not conducive to the further dispersion of magnetic beads in the preservation solution.

Based on this, in some embodiments, a volume percentage of the surfactant in the preservation solution is in a range of 0.5% to 5%, inclusive. A concentration expressed by a percentage of a volume of the solute (in a liquid state) to a total volume of the solution is called a volume percentage concentration. Here, the volume percentage of the surfactant in the preservation solution is in a range of 0.5% to 5%, inclusive, which means that the percentage of the volume of the surfactant to the volume of the preservation solution is in the range of 0.5% to 5%, inclusive. That is, the volume percentage of the surfactant in the preservation solution may be any value from 0.5% to 5%. For example, the volume percentage of the surfactant in the preservation solution may be 0.5%, 1%, 1.5%, 2%, 2.5%, 3%, 3.5%, 4%, 4.5% or 5%, etc.

In some embodiments, the volume percentage of the surfactant in the preservation solution is 0.5%. It may reduce the flocculation of the surfactant to the greatest extent and improve the monodispersity of the magnetic beads.

In some embodiments, the surfactant is selected from any or combinations of two or more of polysorbate-20 (TWEEN 20), sodium dodecyl sulfate (SDS) and sorbitan fatty acid esters (trade name: Span). In a case where the surfactant is selected from combinations of any two or more of polysorbate-20 (TWEEN 20), sodium dodecyl sulfate (SDS) and sorbitan fatty acid esters, the any two or more surfactants may be mixed in any proportion.

Both TWEEN 20 and sorbitan fatty acid esters are nonionic surfactants containing sorbitol structure. The molecule contains a plurality of hydrophilic groups, which may be miscible with water, methanol, ethanol, isopropanol, propylene glycol, ethylene glycol, etc. Sodium dodecyl sulfate is an anionic surfactant. These surfactants are the most commonly used surfactants and are chemically stable.

In these embodiments, it is found through experiments that these surfactants may be combined in any proportion to maintain a monodisperse effect of the magnetic beads for a long time. For example, the addition amounts of the surfactant is controlled to be in the range of 0.5% to 5% in volume percentage, and the agglomeration ratio of the magnetic beads may be kept below 6% after the magnetic beads are stored in the above preservation solution for 3 months.

In some examples, the volume percentage of TWEEN 20 in the preservation solution is 0.2%, the volume percentage of sodium dodecyl sulfate (SDS) in the preservation solution is 0.25%, and the volume percentage of sorbitan fatty acid esters in the preservation solution is 0.05%. It is found through experiments that, the addition amounts of TWEEN 20, sodium dodecyl sulfate and sorbitan fatty acid esters are respectively kept within the above ranges, the agglomeration ratio of the magnetic beads may be reduced to the greatest extent and the preservation time may be prolonged. For example, after preservation of the magnetic beads in the preservation solution for 3 months, the magnetic beads are hardly agglomerated.

In chemistry, a salt refers to a compound formed by combining a metal ion or an ammonium ion (NH4⁺) with an acid radical ion (anion) through ionic bond(s). A water-soluble salt refers to a salt that can be dissolved in water, and all ions will be dissociated when the water-soluble salt is dissolved in water.

Water-soluble salts may provide an ionic environment for the magnetic beads in water. Through the interaction between charged ions and groups on surfaces of the magnetic beads, the groups on the surfaces of the magnetic beads may be in a relaxed state, thereby avoiding agglomeration due to the interaction between groups of magnetic beads and groups of magnetic beads. The reason is that the water-soluble salts are ionized into cations and anions in water, and according to the mutual attraction characteristics between anions and cations, in a case where the cations of the water-soluble salts and the groups (e.g., carboxyl groups) on the magnetic beads form hydrated ions, the anions of the water-soluble salts surround the hydrated ions. In this way, a layer of anion film may be formed on the surface of the magnetic bead, and the magnetic beads may be kept dispersed by using the electrostatic repulsion between the magnetic beads.

The water-soluble salt may be selected from one of monovalent salts and polyvalent salts or a plurality of combinations of the monovalent salts and the polyvalent salts, which is not specifically limited here.

It will be noted that the addition amounts of water-soluble salts are related to the charge amount of the charged ion of the water-soluble salt. It can be known based on the above mechanism that, in a case where the groups on the surface of the magnetic bead are certain, in order to make the groups on the surface of the magnetic bead are all combined with the charged ion of the water-soluble salt to form hydrated ions, the charged amounts of the charged ions of the required water-soluble salts are the same as the charged amounts of the groups on the surfaces of the magnetic beads, and positive or negative of charges of the charged ions of the required water-soluble salts are opposite to positive or negative of charges of the groups on the surfaces of the magnetic beads. In an example where the group on the surface of the magnetic bead is the carboxyl group, in a case where the water-soluble salt is selected from monovalent salts such as sodium chloride, a carboxyl group and a sodium ion may form a hydrated ion, and it is assumed that the molar concentration of the carboxyl group in the preservation solution is 1 mol/L, the molar concentration of the sodium chloride that needs to be added in the preservation solution is also 1 mol/L. In a case where the water-soluble salt is selected from divalent salts such as magnesium chloride, a magnesium ion and two carboxyl groups may form a hydrated ion, and the molar concentration of the magnesium chloride that needs to be added in the preservation solution is 0.5 mol/L. It can be seen that for monovalent salts, in order to achieve the same technical effect as polyvalent salts, it is necessary to add a high concentration in the preservation solution. However, if the addition concentration is too high, it is easy to cause flocculation in the preservation solution of magnetic beads, which is not conducive to the monodispersion of the magnetic beads.

In some embodiments, the water-soluble salt is selected from one of divalent salts or a plurality of combinations of divalent salts, and in a case where the water-soluble salt is selected from the plurality of combinations of divalent salts, a chemical reaction does not occur between a plurality of divalent salts.

In the divalent salt, a valence of a metal in a positive valence state in the elements is positive divalent, such as magnesium sulfate, magnesium chloride, zinc chloride. Compared with monovalent salt, in a case of the equivalent molar concentration of cations, the charged amounts of cations in divalent salts are more helpful to make the groups on the surfaces of magnetic beads be in the relaxed state. In addition, the chemical reaction does not occur between the plurality of divalent salts, so that the plurality of divalent salts may all exist in ion states, thereby preventing chemical reactions between the various divalent salts from generating precipitates.

In some embodiments, the water-soluble salt is selected from one of calcium salts and magnesium salts or a plurality of combinations of calcium salts and magnesium salts.

For example, the water-soluble salt is selected from one or a combination of calcium chloride and magnesium chloride.

The molar concentration of the water-soluble salt in the preservation solution is not specifically limited. For different preservation solution systems of magnetic beads, the molar concentration of the water-soluble salt in the preservation solution may be reasonably set to avoid agglomeration of magnetic beads.

In some embodiments, the molar concentration of the water-soluble salt in the preservation solution is in a range of 1 mol/L to 3 mol/L, inclusive. That is, the molar concentration of the water-soluble salt in the preservation solution may be any value from 1 mol/L to 3 mol/L, inclusive. For example, the molar concentration of the water-soluble salt in the preservation solution may be 1 mol/L, 1.5 mol/L, 2 mol/L, 2.5 mol/L or 3 mol/L.

It is found through experiments that by keeping the molar concentration of the water-soluble salt in the preservation solution within the above range, the groups on the surfaces of the magnetic beads may be in the relaxed state, and it may prevent excessive concentration of water-soluble salts from causing flocculation in the preservation solution of magnetic beads, thereby maintain the monodisperse effect of the magnetic beads for a long time.

The bacteriostatic agent is a substance that inhibits the growth of bacteria. The bacteriostatic agent may not kill the bacteria, but it can inhibit the growth of the bacteria and stop the bacteria from multiplying too much.

In embodiments of the present disclosure, the bacteriostatic agent may be any substance capable of inhibiting the growth of bacteria, which is not specifically limited here.

In some embodiments of the present disclosure, the bacteriostatic agent is selected from antibiotics. Antibiotics are mainly secondary metabolites or artificially synthesized analogs produced by bacteria, mold or other microorganisms, and are mainly used to treat various diseases caused by bacterial infections or pathogenic microorganisms. They can selectively act on specific links of deoxyribonucleic acid (DNA), ribonucleic acid (RNA) or protein synthesis systems of bacterial cells (or thallus cells), interfere with the metabolism of cells, hinder life activities or make cells stop growth and even die. Therefore, adding the antibiotics in the preservation solution may prevent bacterial infection to the greatest extent and have a good antibacterial effect. In addition, when the preservation solution of magnetic beads is used for the preparation and treatment of downstream products, the preservation solution of magnetic beads may also sterilize bacteria in downstream products, and may be used for aseptic preservation of downstream products.

In some embodiments, the antibiotics are broad-spectrum antibiotics. The broad-spectrum antibiotics refer to drugs with a relatively broad antibacterial spectrum, in short, the drugs that can resist most bacteria. Adding the broad-spectrum antibiotics into the preservation solution may ensure that the preservation solution will not be infected by most bacteria during preservation.

In some embodiments, an antibiotic is selected from one of chloramphenicol, tetracycline and vancomycin, or a plurality of combinations of chloramphenicol, tetracycline and vancomycin. A volume percentage of each antibiotic in the preservation solution is in a range of 0.1% to 0.5%, inclusive.

In a case where the antibiotic is selected from one of chloramphenicol, tetracycline and vancomycin, the volume percentage of this antibiotic in the preservation solution may be any value from 0.1% to 0.5%, inclusive. By considering an example of chloramphenicol being selected as the antibiotic, the volume percentage of chloramphenicol in the preservation solution may be 0.1%, 0.15%, 0.2%, 0.25%, 0.3%, 0.35%, 0.4%, 0.45% or 0.5%. In a case where the antibiotic is selected from the plurality of combinations of chloramphenicol, tetracycline and vancomycin, the volume percentages of different types of antibiotics in the preservation solution may each be any value from 0.1% to 0.5%, inclusive. By considering an example of chloramphenicol and vancomycin being selected as the antibiotic, the volume percentage of chloramphenicol in the preservation solution may be 0.1%, 0.15%, 0.2%, 0.25%, 0.3%, 0.35%, 0.4%, 0.45% or 0.5%, and the volume percentage of vancomycin in the preservation solution may also be 0.1%, 0.15%, 0.2%, 0.25%, 0.3%, 0.35%, 0.4%, 0.45% or 0.5%. Of course, it can be understood that, in a case where chloramphenicol, tetracycline and vancomycin are selected as the antibiotic, the volume percentage of chloramphenicol in the preservation solution may be 0.1%. 0.15%, 0.2%, 0.25%, 0.3%, 0.35%, 0.4%, 0.45% or 0.5%, the volume percentage of tetracycline in the preservation solution may be 0.1%, 0.15%, 0.2%, 0.25%, 0.3%, 0.35%, 0.4%, 0.45% or 0.5%, and the volume percentage of vancomycin in the preservation solution may also be 0.1%, 0.15%, 0.2%, 0.25%, 0.3%, 0.35%, 0.4%, 0.45% or 0.5%.

In these embodiments, one or more antibiotics are selected and a volume percentage of each antibiotic in the preservation solution is controlled to the above range. Since each antibiotic has different functions, in a case where the antibiotic is selected from the plurality of combinations, it may inhibit the growth of different kinds of bacteria.

In some examples, chloramphenicol and vancomycin are selected as the antibiotic, the volume percentage of chloramphenicol in the preservation solution is 0.3%, and the volume percentage of vancomycin in the preservation solution is 0.5%.

In these embodiments, it is found through experiments that by selecting chloramphenicol and vancomycin and limiting the volume percentages of chloramphenicol and vancomycin in the preservation solution to the above ranges, the preservation time of the magnetic beads may be improved to the greatest extent, and may meet actual preservation requirements.

In some embodiments, the preservation solution may further include: ethylenediaminetetraacetic acid (EDTA). EDTA may combine with calcium ions and magnesium ions in the preservation solution to form chelates. Since action of most nucleases and some proteases needs Mg²⁺, EDTA combined with Mg²⁺ may inhibit the enzymolysis reaction of nucleases and proteases.

The addition amounts of EDTA are not specifically limited, as long as EDTA may combine with free calcium ions and magnesium ions in the preservation solution to form chelates, so as to avoid excessive free magnesium ions from participating in the enzymolysis reaction of most nucleases and some proteases.

In some embodiments, a molar concentration of EDTA in the preservation solution is in a range of 0.5 mmol/L to 20 mmol/L, inclusive. That is, the molar concentration of EDTA in the preservation solution may be any value from 0.5 mmol/L to 20 mmol/L. For example, the molar concentration of EDTA in the preservation solution may be 0.5 mmol/L, 1 mmol/L, 1.5 mmol/L, 2 mmol/L, 2.5 mmol/L, 3 mmol/L, 3.5 mmol/L, 4 mmol/L, 4.5 mmol/L, 5 mmol/L, 5.5 mmol/L, 6 mmol/L, 6.5 mmol/L, 7 mmol/L, 7.5 mmol/L, 8 mmol/L, 8.5 mmol/L, 9 mmol/L, 9.5 mmol/L, 10 mmol/L, 10.5 mmol/L, 11 mmol/L, 11.5 mmol/L, 12 mmol/L. 12.5 mmol/L. 13 mmol/L, 13.5 mmol/L, 14 mmol/L, 14.5 mmol/L, 15 mmol/L, 15.5 mmol/L, 16 mmol/L, 17.5 mmol/L, 18 mmol/L, 18.5 mmol/L, 19 mmol/L, 19.5 mmol/L or 20 mmol/L.

In these embodiments, by limiting the molar concentration of EDTA within the above range, it may play the role of anti-enzymolysis, and it may also play a bacteriostatic role in a case of excessive EDTA.

In some embodiments, the preservation solution further includes a buffer agent, and an additive amount of the buffer agent causes a pH value of the preservation solution is in a range of 7.5 to 8.5, inclusive.

In these embodiments, by adding the buffer agent, the pH value of the preservation solution may be maintained within the range of 7.5 to 8.5, thereby avoiding a large change in pH value of the preservation solution, which is not conducive to the application of the preservation solution. That is, the pH value of the preservation solution may be any value in the range of 7.5 to 8.5.

The above buffer agent may be any buffer agent that can maintain the pH value of the preservation solution within the range of 7.5 to 8.5, and is not specifically limited here.

In some embodiments, Tris-EDTA buffer (TE) is selected as the buffer agent. TE buffer is formulated from Tris (tris(hydroxymethyl)methyl aminomethane) and EDTA (ethylenediaminetetraacetic acid), and is mainly used to dissolve nucleic acids and can stably preserve deoxyribonucleic acid (DNA) and ribonucleic acid (RNA). TE buffer is a solution that can resist pH changes when small amounts of acid or base are added.

In some embodiments, a molar concentration of the buffer in the preservation solution is in a range of 0.1 mmol/L to 10 mmol/L, inclusive. That is, the molar concentration of the buffer agent in the preservation solution may be any value from 0.1 mmol/L to 10 mmol/L. For example, the molar concentration of the buffer agent in the preservation solution may be 0.1 mmol/L, 0.2 mmol/L, 0.5 mmol/L, 1 mmol/L, 1.5 mmol/L, 2 mmol/L, 2.5 mmol/L, 3 mmol/L, 3.5 mmol/L, 4 mmol/L, 4.5 mmol/L, 5 mmol/L, 5.5 mmol/L, 6 mmol/L, 6.5 mmol/L, 7 mmol/L, 7.5 mmol/L, 8 mmol/L, 8.5 mmol/L, 9 mmol/L, 9.5 mmol/L or 10 mmol/L.

Some embodiments of the present disclosure provide a preservation method of magnetic beads, which includes:
dispersing the magnetic beads in the preservation solution of magnetic beads as described above.

For example, a certain amount of magnetic beads may be weighed, the magnetic beads are placed in the preservation solution of magnetic beads, and the magnetic beads are dispersed under stirring conditions. Magnetic beads include ferroferric oxide, and polyethylene glycol (PEG) and organic molecules (the organic molecules are bonded to PEG, and an outer layer of the organic molecule has specific groups) wrapped on the surface of ferroferric oxide.

In order to make the magnetic beads fully dispersed in the preservation solution, optionally, the concentration of the magnetic beads in the preservation solution is 10 mg/mL.

The size distribution variance of the magnetic beads is less than 5%, that is, the particle sizes of the magnetic beads are basically the same. In this case, the magnetic beads have monodispersity.

In these embodiments, the magnetic beads are dispersed in the above preservation solution of magnetic beads for preservation. The surfactant in the preservation solution plays a role of dispersion and stabilization, the water-soluble salt plays a role of dispersing the magnetic beads, and the bacteriostatic agent plays an antibacterial role. Compared with the related art in which only a surfactant and a buffer agent are added, the addition of the water-soluble salt may maintain the monodispersity of the magnetic beads to prevent agglomeration of the magnetic beads, and the addition of the bacteriostatic agent may prevent the formation of bacteria during preservation. Therefore, the magnetic beads preserved based on the above preservation solution may be preserved for a longer time.

In some embodiments, the preservation temperature of the magnetic beads is in a range of 4° C. to 8° C., inclusive.

In these embodiments, refrigerated preservation may further prolong the preservation time of the magnetic beads.

In order to objectively evaluate technical effects of the embodiments of the present disclosure, the following will be exemplarily described in detail through a comparative example and experimental examples.

For the following comparative example and experimental examples, the experimental methods used are conventional methods unless otherwise specified.

For the following comparative example and experimental examples, materials and reagents used may be purchased from commercial sources unless otherwise specified.

In the following comparative example and experimental examples, concentrations of magnetic beads in the preservation solutions are all 10 mg/mL, and the preservation temperatures are all 4° C. The compositions of the preservation solutions are shown in Table 1 below.

TABLE 1

| | | | | Water- | |
|---|---|---|---|---|---|
| | Surfactant/ | Buffer agent/ | EDTA/ | soluble salt/ | |
| | volume | molar | molar | molar | Bacteriostat |
| | percentage | concentration | concentration | concentration | agent/volume |
| name | (%) | (mmol/L) | (mmol/L) | (mol/L) | percentage (%) |
| Comparative | (TWEEN) | 1 | 0.5 | / | / |
| Example | 0.5% | | | | |
| Experimental | (TWEEN 20 + | 2 | 0.5 | (magnesium | (chloramphenicol + |
| Example 1 | SDS + Span) | | | chloride) | vancomycin) |
| | 0.5% + 0.5% + 3% | | | 1 | 0.5% + 0.3% |
| Experimental | (TWEEN 20) | 1 | 1 | (magnesium | (chloramphenicol) |
| Example 2 | 1% | | | chloride) | 0.5% |
| | | | | 1.5 | |
| Experimental | (SDS) | 3 | 1.5 | (magnesium | (chloramphenicol + |
| Example 3 | 2% | | | chloride) | vancomycin) |
| | | | | 2 | 0.5% + 0.3% |
| Experimental | (Span) | 1.5 | 1 | (calcium | (Vancomycin) |
| Example 4 | 3% | | | chloride) | 0.3% |
| | | | | 1 | |
| Experimental | (TWEEN 20 + | 0.5 | 1.5 | (calcium | (chloramphenicol + |
| Example 5 | SDS) | | | chloride) | vancomycin) |
| | 0.5% + 0.5% | | | 1.5 | 0.5% + 0.3% |
| Experimental | (TWEEN 20 + | 0.2 | 0.8 | (magnesium | (Vancomycin) |
| Example 6 | SDS + Span) | | | chloride + | 0.5% |
| | 0.5% + 0.5% + 3% | | | calcium | |
| | | | | chloride) | |
| | | | | 1 + 1 | |

In Table 1, “/” indicates that there is no such component in the preservation solution, that is, in the comparative example, the preservation solution does not contain a water-soluble salt and a bacteriostatic agent. In experimental example 1, “(TWEEN 20+SDS+Span) 0.5%+0.5%+3%” means that the surfactant includes TWEEN 20, SDS and Span, and the volume percentage of TWEEN 20 in the preservation solution is 0.5%, the volume percentage of SDS in the preservation solution is 0.5%, and the volume percentage of Span in the preservation solution is 3%. Similarly, “(chloramphenicol+vancomycin) 0.5%+0.3%” means that the bacteriostatic agent includes chloramphenicol and vancomycin, the volume percentage of chloramphenicol in the preservation solution is 0.5%, and the volume percentage of vancomycin in the preservation solution is 0.3%. “(magnesium chloride+calcium chloride) 1+1” means that the water-soluble salt includes magnesium chloride and calcium chloride, and the molar concentration of magnesium chloride in the preservation solution and the molar concentration of calcium chloride in the preservation solution are each 1 mol/L.

Sampling is carried out every other month, observed under a microscope, the numbers of agglomerated magnetic beads in samples of comparative example and experimental example 1 to experimental example 6 are recorded, and the agglomeration ratios are calculated until they have been preserved for 6 months.

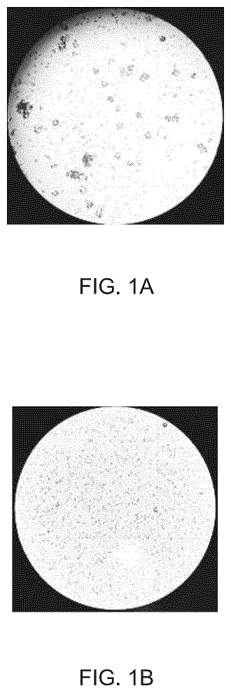

FIG. 1A is a dispersion diagram of magnetic beads in comparative example after preservation for 6 months. FIG. 1B is a dispersion diagram of magnetic beads in experimental example 1 after preservation for 6 months. As shown in FIGS. 1A and 1B, after preservation for 6 months, most magnetic beads in comparative example are agglomerated, whereas only a small part of magnetic beads in experimental example 1 are agglomerated, and most part thereof are in a monodisperse state.

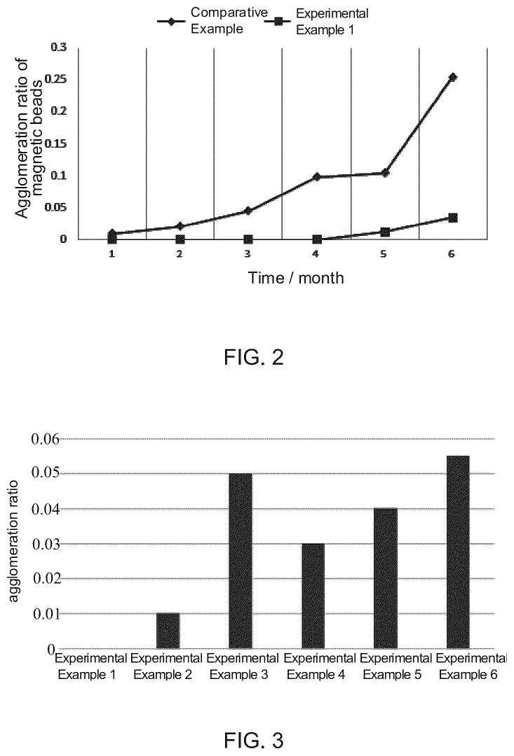

FIG. 2 is a broken line graph of variations of agglomeration ratio of magnetic beads in comparative example and experimental example 1 during preservation. It can be seen from FIG. 2 that with the prolongation of preservation time, a large degree of agglomeration occurs in the magnetic beads in comparative example after preservation for 3 months, and the agglomeration ratio of the magnetic beads increased sharply after preservation for 5 months. However, in experimental example 1, the magnetic beads are only less agglomerated after preservation for 5 months, and the agglomeration ratio of the magnetic beads is still in a relatively small range after preservation for 6 months.

FIG. 3 is a comparison diagram of agglomeration ratio of magnetic beads after preservation for 3 months in experimental example 1 to experimental example 6. It can be seen from FIG. 3 that in experimental example 1 to experimental example 6, after preservation for 3 months, the agglomeration ratios of the magnetic beads are all less than 6%. It can be seen that the preservation solution provided by the present disclosure may maintain the monodispersity of the magnetic beads, thereby greatly prolonging the preservation time of the magnetic beads and meeting the current application requirements. Moreover, it can be seen from FIG. 3 that after preservation for 3 months in experimental example 1, the magnetic beads are hardly agglomerated. It can be seen that by selecting various components and setting the concentration of the selected component in the preservation solution reasonably, the agglomeration of magnetic beads may be reduce to the greatest extend, and unexpected technical effects may be obtained. Moreover, it can be seen that the preservation effect of the preservation solution is a result of the synergistic effect of all components, and have a broad application prospect.

The foregoing descriptions are merely specific implementations of the present disclosure, but the protection scope of the present disclosure is not limited thereto, any changes or replacements that a person skilled in the art could conceive of within the technical scope of the present disclosure shall be included in the protection scope of the present disclosure. Therefore, the protection scope of the present disclosure shall be determined by the protection scope of the claims.

1. A preservation solution of magnetic beads, comprising: a surfactant, a water-soluble salt and a bacteriostatic agent.

2. The preservation solution of magnetic beads according to claim 1, wherein
the water-soluble salt is selected from one of divalent salts or a plurality of combinations of the divalent salts.

3. The preservation solution of magnetic beads according to claim 2, wherein
the water-soluble salt is selected from one of calcium salts and magnesium salts or a plurality of combinations of the calcium salts and the magnesium salts.

4. The preservation solution of magnetic beads according to claim 3, wherein
the water-soluble salt is selected from one or a combination of calcium chloride and magnesium chloride.

5. The preservation solution of magnetic beads according to claim 1, wherein
a molar concentration of the water-soluble salt in the preservation solution is in a range of 1 mol/L to 3 mol/L, inclusive.

6. The preservation solution of magnetic beads according to claim 1, wherein
the bacteriostatic agent is selected from antibiotics.

7. The preservation solution of magnetic beads according to claim 6, wherein
the antibiotics are broad-spectrum antibiotics.

8. The preservation solution of magnetic beads according to claim 7, wherein
an antibiotic is selected from one of or a plurality of combinations of chloramphenicol, tetracycline and vancomycin, and a volume percentage of each antibiotic in the preservation solution is in a range of 0.1% to 0.5%, inclusive.

9. The preservation solution of magnetic beads according to claim 1, wherein
a volume percentage of the surfactant in the preservation solution is in a range of 0.5% to 5%, inclusive.

10. The preservation solution of magnetic beads according to claim 1, further comprising ethylenediaminetetraacetic acid.

11. The preservation solution of magnetic beads according to claim 10, wherein
a molar concentration of the ethylenediaminetetraacetic acid in the preservation solution is in a range of 0.5 mmol/L to 20 mmol/L, inclusive.

12. The preservation solution of magnetic beads according to claim 1, further comprising a buffer agent, an additive amount of the buffer agent causes a pH value of the preservation solution is in a range of 7.5 to 8.5, inclusive.

13. A preservation method of magnetic beads, comprising:
dispersing the magnetic beads in the preservation solution of magnetic beads according to claim 1.

14. The preservation method of magnetic beads according to claim 13, wherein
a preservation temperature of the magnetic beads is in a range of 4° C. to 8° C., inclusive.

15. The preservation solution of magnetic beads according to claim 1, wherein the water-soluble salt is selected from a plurality of combinations of divalent salts, and a chemical reaction does not occur between a plurality of divalent salts.

16. The preservation solution of magnetic beads according to claim 5, wherein the bacteriostatic agent is selected from antibiotics.

17. The preservation solution of magnetic beads according to claim 1, wherein the surfactant includes an ionic surfactant or a nonionic surfactant.

18. The preservation solution of magnetic beads according to claim 17 wherein the surfactant is selected from any or combinations of two or more of polysorbate-20, sodium dodecyl sulfate and sorbitan fatty acid esters.

19. The preservation solution of magnetic beads according to claim 5, wherein a volume percentage of the surfactant in the preservation solution is in a range of 0.5% to 5%, inclusive.

20. The preservation solution of magnetic beads according to claim 12, wherein a molar concentration of the buffer agent in the preservation solution is in a range of 0.1 mmol/L to 10 mmol/L, inclusive.
